(19) United States
(12) Patent Application Publication
(10) Pub. No.: US 2001/0010022 A1
(43) Pub. Date: Jul. 26, 2001

(54) Medical product, method for its manufacture and use

(76) Inventors: Martin Dauner, Esslingen (DE); Heinrich Planck, Nuertingen (DE); Carsten Linti, Stuttgart (DE)

Correspondence Address: NATH & ASSOCIATES, Sixth Floor, 1030 15th Street, N.W., Washington, DC, 20005, US

(21) Appl. No.: 09/728,483

(22) Filed: Dec. 4, 2000

(30) Foreign Application Priority Data
Dec. 8, 1999 (DE) 19959088.5

(51) Int. Cl. A61F 2/02

(52) U.S. Cl. 623/23.71; 623/23.76

(57) ABSTRACT

A medical product is made available having a melt-blown fibrous structure of biocompatible polymer material in the form of a three-dimensional shaped article with a porous structure aiding cell growth. It can be used in human and/or veterinary medicine, as an implant or extracorporeal organ replacement.

### DESCRIPTION

The present invention relates to a medical product, a method for its manufacture and its use in medicine.

Biocompatible materials are required in medical technology for the production of implants and for organ replacements. For special uses such as vascular prostheses or cartilage replacement the implant materials must be constructed in a desired form or shape, e.g. a hollow article or body. So that the implant can fulfil in optimum manner its function in the body, the implant must grow in in a satisfactory manner and is preferably completely colonized by body cells.

Implants manufactured by conventional plastics processing technology can admittedly be manufactured with a desired shape, but have an unstructured surface and consequently tend to be cell-repelling in the environment of a living body, which impedes the growing in of body cells.

Implants produced form fibres or yarns using textile procedures in the form of woven and knitted fabrics or nonwovens have a surface structure and porosity. Once again the shaping is restricted by the manufacturing procedure such as weaving, knitting, needling, etc. Moreover, in the case of hollow articles, such as tubular products, problems often arise with stiffness, so that the lumen collapses if the internal pressure drops.

The problem of the present invention is to provide a medical or medicotechnical product, which is made from biocompatible polymer material, which can be constructed with little effort and at reasonable cost in a random shape and which favours cell growth when used in medicine.

This problem is solved by a medical product with a melt-blown fibrous structure of biocompatible polymer material in the form of a three-dimensional shaped article with a porous structure, which aids cell growth.

In the melt-blown method a thermoplastic polymer suitable for fibre formation is forced through a nozzle head, which has a very large number, usually several hundred small apertures generally with a diameter of approximately 0.4 mm. Hot gas flows at approximately 100 to 360° C. passing out and converging around the nozzle head, as a function of the polymer used, carry with them the fibrous, extruded polymer, so that it is simultaneously stretched. Very fine fibres with a diameter of a few micrometers are obtained. In a powerful gas flow the spinning-fresh, stretched fibres are supplied to a collecting device, where a fine fibre layer is formed as an air-intermingled, bonding nonwoven. The adhesion of the staple fibres in the fibre composite is due to the combined action of entangling and bonding of the still melt-warm, not completely solidified fibres.

In an embodiment of the medical product according to the invention it can be in the form of a hollow article. Preferably the medical product is in the form of a tubular article. An example of such a tubular article is provided by implants for replacing vessels for transporting body fluids and tubular body organs such as the esophagus or trachea.

The medical product according to the invention can, in another embodiment, be a free form. An example of such a free form is the simulation of the external ear as a replacement for a missing, endogenic ear.

Advantageously the medical product according to the invention can be constructed in the form of several superimposed layers, i.e. the shaped article according to the invention has in the cross-section of a material a layer structure of melt-blown fibres. In such a layer structure it is possible to use different polymers. In addition, the fibres used can differ as regards diameter and/or characteristics. The individual layers can also differ as regards porosity, pore size and/or pore volume. Thus, through a suitable choice of the layer structure it is possible to vary functional characteristics, such as e.g. degradability or blood compatibility.

In the case of a free form the material can in cross-section have a layer structure. It is also possible to superimpose flat layers of a melt-blown fibrous structure to give a three-dimensional structure. The construction of a layer structure with melt-blown fibres is particularly simple, because further fibrous layers can be applied by melt-blown stages and form a composite in the melting heat with the underlying layer.

The medical product with the melt-blown fibrous structure can advantageously have functional elements.

The medical product can also have reinforcing elements, e.g. in the form of reinforcing rods, reinforcing rings, reinforcing clasps, reinforcing spirals, reinforcing fibres, textile structures, etc., either alone or combined with one another. Preferred materials for the reinforcing elements are biocompatible polymers, biocompatible metals, biocompatible ceramics and/or biocompatible composites.

In particular, such reinforcing elements can be introduced radially. It is also possible to axially, circumferentially introduce such reinforcing elements. The medical product according to the invention is with particular advantage characterized by being self-supporting.

Using a multistage melt-blown process, as is described hereinbefore for the construction of layer structures, the reinforcing elements can be easily and reliably introduced into the medical product. Firstly one or more base layers of melt-blown fibrous material are formed, following the mounting of one or more reinforcing elements and then in one or more stages polymer fibrous material is applied in accordance with the melt-blown method. The reinforcing elements can be fastened in this way in the medical product and embedded in the biocompatible polymer material.

It is also possible to incorporate membranes, e.g. capillary membranes. Such a medical product can advantageously act as an immunological separating membrane. It simultaneously permits the transport of small molecules, such as is e.g. advantageous for nutrient transport. It is also possible to use a membrane for gassing, e.g. with oxygen or carbon dioxide.

To aid cell growth a porous structure is of particular significance in the medical product. The medical product according to the invention is more particularly characterized in that the pore size of the belt-blown fibrous structure can be more than 3 micrometers (>3 μm). In particular, the pore size of the medical product according to the invention can be 10 to 300 μm. In a particularly preferred embodiment the pore size in the medical product can be 20 to 100 μm. According to the invention the melt-blown fibrous structure can have a porosity of 50 to 99%.

The medical product according to the invention can have a strength per unit area which is conventional for the selected polymer and structure. If the medical product according to the invention is to be used for cell colonization, strength plays only a minor part.

In an embodiment of the medical product according to the invention the polymer material under physiological conditions is at least partly, but preferably substantially non-resorbable.

The polymer material for the medical product according to the invention can be chosen from the group of thermoplastic polymers, e.g. polyurethane (PU), polyethylene terephthalate (PET), polybutylene terephthalate (PBT), polyether ketones (PEK), polysulphones (PSU), polypropylene (PP), polyethylene (PE), copolymers, terpolymers and/or mixtures thereof. It is also possible to use elastomeric polymers.

In another embodiment of the medical product according to the invention the polymer material under physiological conditions can be at least partly resorbable. In particular, through the choice of different resorbable polymers it is possible to vary the degradation and/or resorption behaviour. Through the choice of the structure of the medical product according to the invention it is also possible to vary the degradation and/or resorption behaviour.

The polymer material for the medical product according to the invention can be chosen from the group of resorbable thermoplastic polymers comprising polyglycolide, polylactide, polycaprolactone, trimethylene carbonate, resorbable polyurethanes, copolymers, terpolymers and/or mixtures thereof.

In a preferred embodiment the medical product can be characterized in that the melt-blown fibrous material is at least partly and preferably substantially resorbable, whereas introduced functional elements and/or reinforcing elements are only partly resorbable.

It may be necessary to use two or more different polymer materials to obtain specific characteristics. For this purpose fibres or particles can be blown into the air flow. In another variant different polymers can be mixed in the extruder to a so-called blend.

In another construction it is possible to use a biocomponent or multicomponent melt-blow spinning head, in which two or more polymer melts are processed simultaneously. Prior to leaving the nozzle (capillary bore) the melt flows can be combined. Alternatively they can be separately blown through different nozzles (capillary bores), which are arranged in alternating manner or in series. Preferably polymers having different degradation behaviour characteristics are processed together. It is also possible to jointly obtain different surface characteristics, such as e.g. hydrophobic and hydrophilic. One polymer component can also be in binder form.

In an embodiment of a multilayer medical product according to the invention substantially all the layers can be formed from melt-blown fibrous material.

In another embodiment of a multilayer, medical product according to the invention at least one layer can have a different structure. For example, the layers can differ in the degree of their porosity and/or pore diameter.

This makes it possible to influence the accessibility of the fibrous layer structure for cells. Thus, high porosity layers of 30 to 300 μm pore size permit a growing through of body tissue, macroporous layers of 3 to 30 μm pore size a growing in/on of body tissue, microporous layers of <3 μm are used for cell selection and nanoporous layers of <0.2 μm pore size are bacterial filters. In this way a layer can be permeable for cells, receive cells in its pore space or can only be surface-affected with cells.

When used in medical technology, it is advantageous for the medical product with melt-blown fibrous structure to be permeable for nutrients and optionally low molecular weight metabolic products, but on a side exposed to contamination conditions, pathogen penetration is impossible.

In another embodiment of a multilayer medical product according to the invention at least one layer is not formed from melt-blown fibrous material. It is e.g. possible to introduce a fabric produced according to other textile methods, such as a woven or knitted fabric or also a semipermeable film layer such as a polymer or metal layer. Such a differently structured layer can e.g. be provided for reinforcement purposes and/or in barrier form.

Advantageously the medical product according to the invention can comprise melt-blown fibrous materials with fibres having a diameter of 0.1 to 100 μm, particularly 5 to 50 μm. Such fibres are characterized by a cross-sectional area of less than 1 μm² to more than 200 μm².

According to the invention medical agents can be incorporated into the medical product. Examples of such medical agents are medicaments, diagnostics, antimicrobial agents, growth factors, contrast materials, hemostatics, hydrogels or superadsorbers.

The present invention also provides a method for the manufacture of a medical product according to a melt-blown method from biocompatible polymer material so as to provide a three-dimensional shaped article with a porous structure aiding cell growth.

According to the invention a three-dimensional article can be shaped in a building up process. Advantageously the method according to the invention is characterized in that for the production of the medical product use is made of a mould, particularly a female mould, which is at least partly filled by melt-blown fibres.

In another embodiment the method according to the invention can be characterized in that for the medical product a coarsely porous support structure, e.g. a lattice structure, is at least partly filled with melt-blown fibres.

In another embodiment the method according to the invention can be characterized in that the medical product is built up at least partly with melt-blown fibres on a preformed hollow shape, e.g. a tubular shape.

In all the method variants fibres produced according to the melt-blown method can be applied in one or more layers. The individual layers can have the same or different thicknesses. Layers can also be applied with different arrangement patterns.

The melt-blown method is particularly advantageous for the manufacture of the medical products according to the invention, because it is possible to process virtually all thermoplastics, including difficultly soluble polymers such as polyethylene terephthalate, polypropylene or polyglycolic acid. In addition, no solvents, additives or other chemical adjuvants are required, which when using the product in medicine could be harmful for the patient.

A medical product of biocompatible polymer material formed from melt-blown fibrous material, which is constructed in the form of a three-dimensional shaped article and has a porous structure aiding cell growth is used in human and/or veterinary medicine. In an embodiment the medical product according to the invention can be used as an implant. The implant advantageously has the three-dimensional shape of a body part to be replaced. A particularly preferred example of the use as an implant is a tracheal prosthesis for the replacement of the trachea of the patient. Medical products for implantation in a human or animal patient can be produced in advantageous manner in the desired shape and with the dimensions adapted to the particular patient. Preferably the medical product according to the invention can be used for the in vitro and/or in vivo colonization with cells. For example, the prefabricated medical product can be colonized in vitro with the cells of the patient. The implant is then inserted in the patient. This leads to a better growing in, faster healing and fewer complications.

In another embodiment the medical product according to the invention can be used as an extracorporeal organ replacement. A particularly preferred example of use is that in a liver reactor for the replacement of a non-functioning liver outside the body of the patient. Non-resorbable polymers are preferably used in this case.

Further features and details of the invention can be gathered from the following description of preferred embodiments in the form of examples. The individual features can be implemented singly or in the form of combinations. The examples merely serve to illustrate the invention and the latter is in no way restricted thereto.

The examples refer to the accompanying drawings, wherein show:

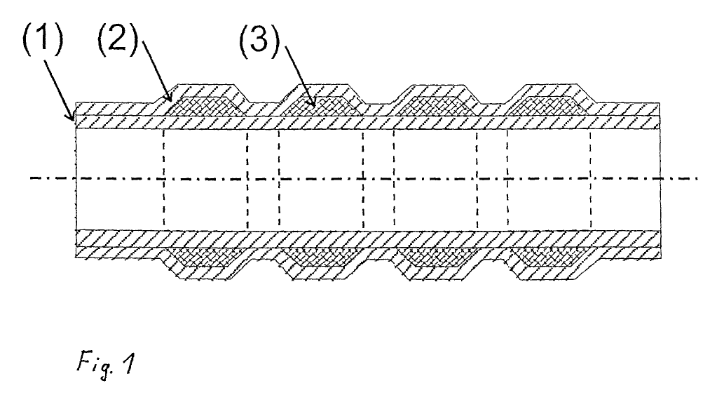

FIG. 1 A longitudinal section through a tracheal prosthesis with the melt-blown fibrous structure according to the invention as the inner and outer fibrous material layer with incorporated reinforcing clasps.

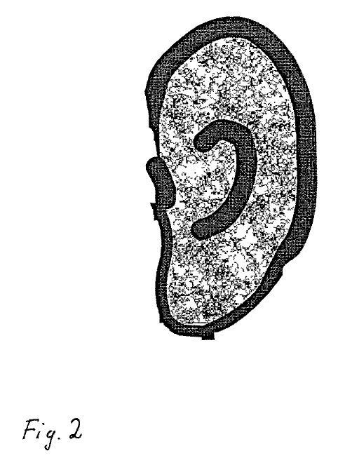

FIG. 2 A diagrammatic representation of a human external ear simulated from inventive melt-blown fibrous structure.

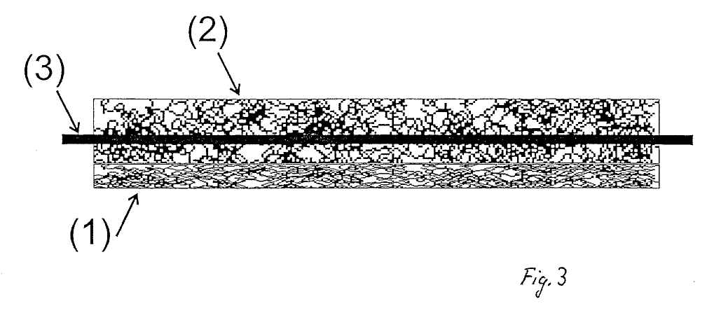

FIG. 3 A diagrammatic representation of a liver reactor inlayer with the inventive melt-blown fibrous structure with incorporated capillaries for gas exchange, a coarsely porous structure for receiving hepatocytes and a finely porous structure for metabolic assistance.

### EXAMPLE 1

### Tracheal prosthesis

For a trachea to be implanted in a patient a tubular hollow structure with horseshoe-shaped reinforcing clasps is produced in accordance with a multistage melt-blown method.

In the attached FIG. 1 reference numeral 1 represents an inner layer of melt-blown fibrous material, 2 an outer layer of melt-blown fibrous material and 3 incorporated reinforcing clasps.

With the aid of a tubular screening device of suitable dimensions firstly a microporous inner wall structure is formed. Then individual horseshoe-shaped reinforcing clasps made from plastic such as e.g. PUR, PET or PP are applied and bonded to the inner layer. Subsequently the macroporous outer layer is applied in accordance with the melt-blown method.

For the first layer polyurethane with a melting point of 180° C. and with a volume flow of 9.6 cm³/min is forced through the nozzle capillaries. For the blowing air heating takes place to 250° C. and at 5.5 bar a volume flow of 45 Nm³/h is produced. The fibrous structure has a porosity of 83% with pores having the size 11 to 87 μm (mean value 30 μm).

For the second layer polyurethane is melted at 180° C. and with a volume flow of 9.6 cm³/min is forced through the capillaries of a nozzle. For the blowing air heating takes place to 230° C. and at 4.75 bar a volume flow of 38 Nm³/h is produced. The melt-blown structure obtained has a porosity of 84% with pores of 16 to 300 μm (mean value 82 μm).

The clasps are such that they only reinforce 270° of the circumference of the prosthesis and leave the remainder free. The latter faces the esophagus in the patient and as a result of its flexibility allows a better swallowing function.

The finely porous inner layer permits a nutrient exchange with the environment, here the respiratory air, but is impermeable to bacteria with which the air could be contaminated. On the side facing the body is located the coarsely porous outer layer of the tracheal prosthesis, which aids an easy growing in of body tissue. In another embodiment the inner layer can be intended for colonization with ciliated epithelium.

In the case of a tracheal prosthesis particular importance is attached to the dimensional stability, flexural rigidity and torsional stiffness. Mechanical characteristics and pore characteristics of structures produced according to the melt-blown method are given in the following table 1.

TABLE 1

| | | | | Breaking | | |
|---|---|---|---|---|---|---|
| | Breaking | | Elongation | strain/ | Pore | Average |
| | strain | Standard | at break | density | volume | pore size |
| Material | [N/mm²] | deviation | [%] | [N * cm/g] | [%] | [μm] |
| Polyurethane | 1.27 | 0.17 | 351 | 482.9 | 77.0 | 19.0 |
| Polyurethane | 1.01 | 0.21 | 368 | 289.9 | 69 | 30 |
| Polyurethane | 0.53 | 0.05 | 300 | 212.2 | 78 | 44 |
| Polyurethane | 0.48 | 0.15 | 267 | 202.6 | 79 | 55 |
| Polyurethane | 0.57 | 0.07 | 323 | 172.2 | 71 | 79 |
| PGA | 0.06 | 0.00 | 43 | 60.4 | 94 | 27 |
| PGA | 0.07 | 0.01 | 53 | 79.4 | 94 | 43 |
| P-L-LA | 0.24 | 0.02 | 47 | 378.0 | 95 | 28 |

### EXAMPLE 2

### External ear prosthesis

Severe psychogenetic disorders arise through congenital or acquired defects extending to the complete lack of the exterior ear. Therapy up to now has used complicatedly cut autotransplants from costal arches. This can be assisted by in vitro tissue growth. A framework is required with the shape of the ear to be grown and in which the cartilage cells can be reorganized.

Such a framework structure can be designed in a particularly advantageous manner according to the melt-blow method, because the fibres can be directly blown into or onto a corresponding shape. The incident airflow is sucked off, as is conventional in the melt-blow method. On the thus formed basic shape of an ear are then applied cartilage cells taken from the patient, which during incubation in vitro completely colonize the ear framework of melt-blown fibres. This leads to a very compatible implant as a result of the use of endogenic cells and which is similar to the natural form. Subsequently the ear prosthesis is introduced into the patient.

### EXAMPLE 3

### Liver reactor

For a liver reactor to be used as an extracorporeal, temporary liver replacement, a multilayer structure of melt-blown fibrous material is formed from non-resorbable polyurethane. On the initially formed, flat nonwoven is placed a capillary membrane and onto it is applied once again melt-blown fibrous material.

In the attached FIG. 3 reference numeral 1 represents a coarsely porous fibrous layer, 2 a finely porous fibrous layer and 3 incorporated capillaries for gas exchange.

For the first layer polyurethane is melted at 180° C. and is pressed with a volume flow of 9.6 cm³/min through the capillaries of a nozzle. The blowing air is heated to 230° C. and at 4.75 bar a volume flow of 38 Nm³/h is produced. The resulting melt-blown structure has a porosity of 84% with pores of 16 to 300 μm (mean value 82 μm). For the second layer polyurethane at the same melting point and with a volume flow of 9.6 cm³/min is pressed through the nozzle capillaries. The blowing air is heated to 250° C. and at 5.5 bar a volume flow of 45 Nm³/h is produced. The fibrous structure has a porosity of 83% with pores of 11. to 87 μm (mean value 30 μm).

The liver reactor comprises two melt-blown fibrous structure layers, namely a coarsely porous layer for receiving hepatocytes and a finely porous layer for the supply with nutrients and through which the cells cannot pass. Capillary membranes are also provided in the coarsely porous layer, which can be used for supply with oxygen, for transporting away CO₂ and also as bile ducts. The porous melt-blown fibrous layer structure is in a closed vessel, through which flows the plasma of the patient to be treated.

1. Medical product with a melt-blown fibrous structure of biocompatible polymer material in the form of a three-dimensional shaped article with a porous structure, which aids cell growth.

2. Medical product according to claim 1, wherein it is in the form of a hollow article.

3. Medical product according to claim 1, wherein it is present as a free form.

4. Medical product according to claim 1, wherein it is constructed in the form of several superimposed layers.

5. Medical product according to claim 1, wherein it has functional elements.

6. Medical product according to claim 1, wherein it has reinforcing elements.

7. Medical product according to claim 1, wherein it is self-supporting.

8. Medical product according to claim 1, wherein the pore size of the melt-blown fibrous structure is >3 μm.

9. Medical product according to claim 8, wherein the pore size is 10 to 300 μm.

10. Medical product according to claim 1, wherein the melt-blown fibrous structure has a porosity of 50 to 99%.

11. Medical product according to claim 1, wherein the polymer material is at least partly non-resorbable under physiological conditions.

12. Medical product according to claim 11, wherein the polymer material is substantially non-resorbable under physiological conditions.

13. Medical product according to claim 1, wherein the polymer material is at least partly resorbable under physiological conditions.

14. Medical product according to claim 4, wherein substantially all the layers are formed from melt-blown fibrous material in the multilayer product.

15. Medical product according to claim 4, wherein at least one layer has a different structure in the multilayer product.

16. Medical product according to claim 4, wherein at least one layer is not formed from melt-blown fibrous material in the multilayer product.

17. Medical product according to claim 1, wherein the melt-blown fibrous material comprises fibres with a diameter of 0.1 to 100 μm.

18. Medical product according to claim 17, wherein the fibre diameter is 5 to 50 μm.

19. Method for the manufacture of a medical product by means of a melt-blow process from biocompatible polymer material to give a three-dimensional shaped article with a porous structure, which aids cell growth.

20. Method for using a medical product from biocompatible polymer material formed from melt-blown fibrous material constructed in the form of a three-dimensional shaped article and having a porous structure aiding cell growth, in at least one of human and veterinary medicine.

21. Method according to claim 20, wherein the medical product is used as an implant.

22. Method according to claim 20, wherein the medical product is used as an extracorporeal organ replacement.
